(19) United States
(12) Patent Application Publication
(10) Pub. No.: US 2009/0061513 A1
(43) Pub. Date: Mar. 5, 2009

(54) Cell sorting and cell cultivation methods

(75) Inventors: Helene Andersson Svahn, Stockholm (SE); Sara Lindstrom, Stockholm (SE)

Correspondence Address: BIRCH STEWART KOLASCH & BIRCH, PO BOX 747, FALLS CHURCH, VA, 22040-0747, US

(73) Assignee: PicoVitro AB, Jonkoping (SE)

(21) Appl. No.: 12/153,185

(22) Filed: May 14, 2008

Related U.S. Application Data
(60) Provisional application No. 60/924,449, filed on May 15, 2007.

(51) Int. Cl. C12N 5/02; C12N 5/00; C12N 5/08

(52) U.S. Cl. 435/366; 435/325; 435/372.1; 435/375

(57) ABSTRACT

A method of cell seeding is provided, which comprises the steps of:
providing a cell sample;
seeding at least one cell of said cell sample into at least one well of a microplate comprising at least 5 wells/cm².

Also provided is a method of cell cultivation comprising the steps of:
seeding at least one cell into a microplate using the method of cell seeding;
incubating the microplate; and
analyzing the contents of at least one well of the microplate.

This application claims priority to U.S. Provisional Application No. 60/924,449, filed May 15, 2007, under 35 U.S.C. 119(e), the entire contents of which are herein incorporated by reference.

### FIELD OF THE INVENTION

The present invention relates to a method of cell seeding, wherein at least one cell is seeded into a microplate comprising a high number of wells/cm², as well as to a method of cell cultivation, wherein at least one cell is seeded, cultivated and analyzed in a microplate.

### BACKGROUND

The methods and platforms for advanced cellomics are important areas of research as the minimum unit of a living system is a single cell. The living human cell is also the ultimate target of all drugs. To fully understand human cells and their biological response to drug therapy, the complex pathways involved in all cellular functions need to be interpreted. Powerful tools for detailed cellular studies are emerging, making it possible to learn more about cell biology. High confidence is put in advanced micro- and nanotechnological tools that, combined with living cells, create so called laboratory-in-a-cell (LIC) and constitute promising approaches for cell access and analysis.

Because of the heterogeneity within a cell population, increased emphasis has been put on analyzing a large number of individual cells and determining the distributions of responses (Mettetal, J. T., Muzzey, D., Pedraza, J. M., Ozbudak, E. M. & van Oudenaarden, A. Predicting stochastic gene expression dynamics in single cells. Proc Natl Acad Sci USA 103, 7304-7309 (2006)). Even in genetically identical populations, phenotypic and behavioral cell-to-cell variations have since long been observed.

A fundamental goal of cell biology is thus to quantify the range of biological responses of individual cells to various physiologically relevant stimuli, as opposed to bulk averages (e.g. Lidstrom, M. E. & Meldrum, D. R. Life-on-a-chip. Nat Rev Microbiol 1, 158-164 (2003)).

Several approaches to single cell analysis have been presented, some of which are accounted for below.

One widely used method of ordering large numbers of single cells into arrays is by micropatterning of surfaces and seeding cells on these controlled patches of extra cellular matrix (ECM) (Kane, R. S., Takayama, S., Ostuni, E., Ingber, D. E. & Whitesides, G. M. Patterning proteins and cells using soft lithography. Biomaterials 20, 2363-2376 (1999)).

The micropatterning approaches, however, are associated with a number of drawbacks. The drawbacks are, among others, disruptive cell tethering and changed cell behavior, the latter limiting the possibilities for long-term analysis. Thus, only short-term analysis is possible. In addition, these approaches often demand adherent cells for successful seeding. Furthermore, several cells instead of single cells easily become attached per micropatterned spot. The usefulness of the micropatterning approaches is consequently limited.

In addition, many attempts to achieve single cell analysis necessarily involve treating all cells in the same manner. This is often the case for microfluidic systems. Moreover, microfluidic systems and similar fluidic devices often give rise to shear stresses, which might decrease the proliferation rate for sensitive cells. This implies a need for alternative methods of analysis which offer the possibility of treating single cells individually.

For some types of single cell analysis, and e.g. cell-to-cell interaction studies, it is important to know the specific position in an analytical system for a specific cell. A specific cell has to be assigned to a specific position, and that specific position and the specific cell have to be addressable later. However, most work on single cell handling techniques involves random positioning of cells, meaning that the cells adhere at any place in the analytical system by simply settling down. Preparing the right dilution of the cell sample, which might involve loss of viability, then becomes critical to give the highest possible single cell occupancy, for example in microwells or on patterned surfaces.

Conventional systems for single cell analysis include capillary electrophoresis (CE) and flow cytometry. By flow cytometry it is possible to obtain the distributions of parameters like cell concentration, cell size, cell shape, protein expression levels, DNA content etc. at the single-cell level in a group (Akerlund, T., Nordstrom, K. & Bernander, R. Analysis of cell size and DNA content in exponentially growing and stationary-phase batch cultures of Escherichia coli. J Bacteriol 177, 6791-6797 (1995)). Time-dependent analysis, in which different cells are sampled at each time point for several minutes, can also be conducted. Flow cytometry, however, reveals limited information on cell content and gives no information on cell proliferation and clone formation capabilities of a cell sample. In addition, although flow cytometry enables analysis of single cells in a group, group average data is obtained instead of individual cell response data.

EP79528 discloses a method for selecting desirable cell clones, which comprises automated cell seeding using a flow cytophotometer. The method comprises adding cultivation media to a microtiter plate, adding single cells to each well of the plate, incubating the microtiter plate, and analyzing the filtrate from the cultivation media for metabolites. The method primarily involves cultivation of prokaryotic cells. It furthermore has low throughput, presumably due to the low number of wells in the plate and the large volumes involved.

Flow cytometry generally provides information about the average properties of cells, revealing how a group of cells changes. Dynamic information on the level of single cells is however not available. Dynamic information requires continuous tracking of the dynamics of specific cells. However, as flow cytometry is an instant method, the sample withdrawn from culture is often discarded after measurement. Flow cytometry thus lacks means for keeping cells in a uniform environment for long-term monitoring. Moreover, it cannot identify a particular cell, especially not after cell division has occurred.

Strategies for acquiring dynamic information without the use of flow cytometry have recently been presented. One example is disclosed in Di Carlo et al, wherein a dynamic single cell culture array with U-shaped trapping structures is presented. The array allows for cultivation of individual adherent cells in a uniform environment (Di Carlo, D., Wu, L. Y. & Lee, L. P. Dynamic single cell culture array. Lab Chip 6, 1445-1449 (2006)). Cell cultivation in this and other similar devices is however often limited to a few hours of cultivation, or at best to one or two days. Such a relatively short time frame will restrict the cell passages to 1-2 cell generations, which consequently might limit the usefulness of the method. Moreover, non-adherent cells, e.g. blood cells, cannot be studied in such an array.

In order to understand cellular processes and behavior, a controlled way of studying high numbers of single cells and their clone formation is greatly needed. Numerous ways of ordering single cells into arrays have previously been described, but methods wherein each cell and/or clone can be directed and continuously addressed to an exact position, cultivated for weeks and treated differently in a high-throughput manner have not been previously described. Accordingly, to progress from temporal observation of a group of cells giving a group average as result, biology needs more single-object approaches.

### DESCRIPTION OF THE INVENTION

One object of the invention is to meet this demand and to provide a new and improved method of controlled cell seeding, wherein from a cell sample a large number of single cells can be seeded into wells of a microplate in a highly reproducible and high-throughput manner.

Another object of the invention is to provide methods of cell cultivation, wherein single cells from a cell sample can be seeded into wells of a microplate and cultivated, enabling short- or long-term study and treatment of cells on the single cell level.

Thus, in a first aspect of the invention, there is provided a method of cell seeding comprising the steps of:
providing a cell sample;
seeding at least one cell of said cell sample into at least one well of a microplate comprising at least 5 wells/cm².

Consequently, the method of cell seeding enables seeding of one or more cells into one or more wells of a microplate having a large well density. In particular, the method enables seeding of single cells into discrete wells, i.e. at least one single cell is seeded into at least one well.

For example, microplates comprising 5 wells/cm²-700 000 wells/cm² can be used with the method. The microplate format and dimension can be any desired format or dimension, provided that the number of wells per cm² is at least 5. If, for example, a microplate with dimensions of a standard 96-well plate is used (128 mm in length and 86 mm in width and with an available well area of 76 cm²), the microplate can comprise from 380 wells to 53 million wells, such as 500-8.5 million wells, such as 1000-100 000 wells, and such as 3000-10 000 wells. Another possible microplate format is represented by the array format, i.e. 76 mm in length and 24 mm in width. If a microplate of array format is used, it can comprise from 91 wells to 12.7 million wells, such as from 91 wells to 2.1 million wells, and such as 500-10 000 wells.

The inventive method enables high-throughput and controlled cell seeding. In this context, high-throughput seeding could for example mean that thousands of single cells can be seeded into discrete wells. Controlled seeding implies that specific single cells are assigned to specific wells and that those specific cells are addressable later. Growth medium may be added to all desired wells simultaneously, prior to cell seeding.

The seeding method according to the invention can for example be performed using any automatic robot equipment.

The seeding method according to the invention can for example be performed using a flow cytometer apparatus, for example a flow cytometer comprising sorting means. By using flow cytometry for cell seeding, the output can be maximized and a high number of cells can be seeded rapidly and with high accuracy. This means that the time needed for seeding a number of single cells into specific wells is very low. In addition, single cell seeding may be performed with a low margin of error. Thus, single cells will indeed be seeded into a majority of the wells assigned for seeding.

The method according to this aspect of the invention can be performed using a type of flow cytometry apparatus known as a fluorescence activated cell sorter (FACS). For example, if cell seeding is performed using a FACS, e.g. a FACSVantage SE Cell Sorter (BD Biosciences), the time needed for seeding of single cells into wells may be one second per cell and well. Furthermore, the desired single cell seeding may be attained for more than 80% of the assigned wells. The use of such an established cell-sorter and analysis instrument provides distinct advantages, including high sensitivity and accuracy. One advantage is the possibility of analyzing cells while they are seeded into the wells and maintaining the possibility for further high-throughput analysis during long time periods.

According to a second aspect of the invention, there is provided a method of cell cultivation comprising the steps of:
seeding at least one cell into a microplate using the method according to the first aspect of the invention;
incubating the microplate; and
analyzing the contents of at least one well of the microplate.

Optional features of the seeding step in this second aspect of the present invention are as defined above in connection with the description of the first aspect of the invention, for example with regard to microplate dimensions and other parameters.

The method of cultivation thus provides rapid and controlled cell seeding, incubation of cells contained in the microplate and further downstream analysis in-plate. Analysis may thus be performed for thousands of single cells contained in discrete wells in parallel.

In the method of cultivation according to the invention, seeding may be performed first and the other steps may be performed in any order. Moreover, in some embodiments of the invention, at least two of the steps of seeding, incubation and analysis may be performed simultaneously. In particular, analysis may be performed while seeding.

In embodiments of the method of cultivation according to the invention, at least one of the steps of incubation and analysis may be repeated at least once.

The method of cultivation according to the invention allows any desired number of cells, for example several thousands of single cells, to be cultivated (or incubated) for any desired time period. Cells can for example be cultivated and continually monitored and analyzed for one month. By seeding single cells into single wells of the microplate, cells can for example be analyzed individually for over 14 generations, ending up with more than 10 000 cells in each well.

Analysis of well contents can be performed directly after cell seeding or at any desired point of time, during incubation or after incubation, and can be repeated at any time. Any standard detection equipment can be used in the analytic step of the inventive method of cultivation. A microscope of any kind (e.g. confocal microscope) can for example be used for manual detection either well by well or several wells at a time in one picture. The number of wells detected in one picture depends on the magnification/object being used. A microscope with a programmable x/y-stage can also be used for automatic detection, for example when analyzing fluorescently labeled cells. Automatic detection here means that a software runs the x/y-stage that moves the plate/array back and forth, which enables photographing (i.e. detection) well by well in high-throughput. An array scanner is another example of a type of detection equipment that can be used in the inventive method. All kinds of fluorescent plate readers can furthermore be used, provided that the point of detection (coordinates in x/y directions) can be controlled (for well by well detection) or run in a scanning mode (for scanning of the whole plate area regardless of the number of wells). It is understood that other types of detection equipment than the types mentioned above can be used in the inventive method.

Analysis of well contents may for example comprise analysis of number of cells, cell appearance, analysis of cell contents, cell products, protein expression, nucleic acids, etc.

The cultivation method according to the invention enables easy location of cells after cloning, or at any desired point in time, because of the possibility of controlled assignment of specific cells to specific wells. This allows for a possibility of collecting dynamic information of the seeded cells. One can, for example, conclude on cells' abilities to proliferate. Cells' abilities to proliferate, sometimes under the influence of affecting drugs, is a tremendously important aspect in life science and drug development, regardless of whether the objects of study are cancer cells, stem cells or other cells.

The methods according to the invention using a microplate provide maintenance of a high cell retention during cultivation and analysis. Problems regarding maintenance of single cells in their original positions of trapping have been discussed earlier, for example in Di Carlo et al, Lab Chip (2006) (supra). Reliability is an important characteristic for any method of cell seeding and cultivation. The inventive method of cultivation thus accomplishes a high degree of reliability in that the method assures that the same single cells in the same wells are studied at different points in time.

In one example of the method of cultivation according to the invention, analytic medium is added to at least one well at any point after the seeding step. In this example, at least one of the steps of incubation, analysis and addition may be repeated at least once. Analytic medium can for example be added before incubation, or at any point of incubation, before or after analysis. Analytic medium can be added at several points during performance of the method and addition can be repeated.

The term “analytic medium” refers to any substance or matter which can be added to at least one well of a microplate in any form. By way of non-limiting example, the analytic medium may comprise chemotherapeutic compounds; proteins; peptides; antibodies; affinity compounds; labeled compounds, for example labeled antibodies or labeled affinity compounds; nucleic acids; particles, such as for example magnetic beads; other compounds having or suspected of having a biological effect (e.g. drugs or drug leads), etc.

Analytic medium can for example be added using an automatic robot equipment. In particular, addition of analytic medium can be performed using a flow cytometer apparatus, such as for example a FACS. A flow cytometer apparatus, such as for example a FACS, may be used for addition of, for example, chemotherapeutic drugs to the wells of a microplate to enable study of cell/clone response to different drugs and drug concentrations in a high-throughput manner. This addition can for example be accomplished by running the flow cytometer in special mode, wherein artificial cells are registered as events which can be sorted out from the flow.

In the context of the present invention, the term “microplate” refers to any substantially planar arrangement of discrete wells or surfaces that can be used for cell cultivation. Microplate refers to, for example, analytical plates, microtiter plates, 96-well plates, multiwell plates, arrays, disks, or other plates used in biology, chemistry and related disciplines. One example of a microplate is disclosed in U.S. Pat. No. 6,037,171, the disclosure of which is hereby incorporated by reference.

One example of a microplate for use in the methods according to the invention comprises wells having tilted walls. Tilted well walls in this context means that the well opening area is larger than the well bottom area. The well walls thus form an acute angle with a plane parallel to the microplate bottom. For example, the walls are tilted with an angle within the range of 20-85°, such as for example 40-65°, in particular 50-60°. One example of a microplate has walls which are tilted at an angle of 54.7°. Tilted walls facilitates seeding of one or more cells, whereby the method of seeding provides improved reproducibility and reliability compared to previously known methods.

If, for example, a microplate with well walls tilted at an angle of 54.7° is used, the well density may be from 5 wells/cm² to 112 000 wells/cm². If for example the microplate with tilted walls as above further has the dimensions of a standard 96-well plate (128 mm×86 mm), the microplate can comprise from 380 wells to 8.5 million wells, such as 500-100 000 wells, such as 1000-10 000 wells, and such as 3000-10 000 wells.

Another example of a microplate for use in the methods according to the invention comprises wells having straight walls, i.e. the angle between the well walls and a plane parallel to the microplate bottom is 90°. Straight well walls enables a microplate comprising a higher well density compared to a microplate having tilted walls. A microplate having straight well walls can for example comprise 5-700 000 wells/cm².

In some embodiments of the invention, the microplate is thin and flat, i.e. the microplate thickness is low and the microplate is planar. The microplate thickness may for example be within the range of 400-2000 μm. One example of a microplate for use in the inventive methods has a plate thickness of 1000 μm. Low plate height provides for improved detection and imaging when analyzing well contents, for example the possibility of coming closer to the sample with an objective in both upright and inverted microscopy. The center-to-center distance between wells in the microplate describes the closeness of the centers of two adjacent wells. The smaller the center-to-center distance, the larger the number of wells in the microplate. The center-to-center distance of wells may for example be within the range of 12-1500 μm, such as for example 30-1500 μm, and such as 900-1500 μm.

The methods according to the invention provide seeding and cultivation in wells of a microplate. In some embodiments of the invention, the wells of the microplate may be small, a single well for example comprising a volume from 1 pl to 200 μl, such as from 2 pl to 2000 nl, and such as from 150-500 nl. The wells may have any shape, for example circular shape, angular shape, etc. In some embodiments of the invention, the wells are square-shaped. Then the well size, i.e. the well side wall, may be from 10 μm to 4000 μm, such as for example 20-640 μm, and such as for example 100-500 μm. The wells may thus be optimized for single cell seeding, single cell culture, and single cell detection, since the wells may provide an appropriate volume of growth medium for single cell cultivation as well as a suitable well size for single cell detection.

For seeding and studying less than 1000 cells in the inventive methods, a microplate of array format (76 mm×24 mm) may be used. The microplate format may be adjusted to the amount of cells that will be seeded and studied as well as to the desired detection method. The array format is also a standard format for analysis and detection in for example microscope stages, flow cytometer x/y-stage plate holders, scanners etc. Furthermore, it is possible to scan the whole array in a standard microarray scanner (for example from Agilent Technologies) for fluorescence detection. Scanning of fluorescently labeled cells in this manner takes only a few minutes and the output, i.e. the results from all wells, will be given simultaneously.

In some embodiments of the invention, the microplate comprises at least a bottom plate and a microgrid plate. The microplate may further comprise a semi-permeable top membrane for sealing of the wells after seeding in order to among other things prevent evaporation. The membrane is semi-permeable in order to allow a fluid, for example gas or liquid, to enter and leave the wells. Sealing of the wells with a top membrane helps to keep the very small volumes of growth medium inside the wells. Sealing of the wells also prevents the cells from leaving their wells and thus ensures reliable results regarding cell maintenance when performing the method.

In the microplate for use in the methods according to the invention, the bottom plate can be made of glass or any other suitable material. Furthermore, the microgrid plate can be made of silicon, or any other suitable material. Also, the top membrane can be made of polydimethylsiloxane (PDMS) or any other suitable material.

In some embodiments of the invention, the microplate is formed from a sandwich structure with three levels: a bottom plate, an etched microgrid plate and a semi-permeable top membrane.

The microplate may furthermore have the format of a standard microtiter plate (size 128×86 mm), in order to facilitate implementation in clinical labs and standard instruments. The microplate can for example have an anodically bonded glass bottom. Alternatively, it can be clamped together with a selected surface to enable surface modifications (e.g. fibronectin, lysine, gelatin, cell seed layers, etc) of the whole bottom plate before assembly into a sandwich structure.

If desired, the microplate may be washed and reused in the inventive methods.

A microplate comprising a flat glass bottom of variable thickness allows all kinds of imaging analysis. Many interesting cellular features can be studied with, for example, confocal microscopy using different labeling techniques and/or different labels for different cells. Cells may for example be fluorescently labeled. Fluorescence labeling can be obtained for example by addition of fluorescently labeled affinity compounds, live/dead cell stain (e.g. Calcein AM), nuclear dye (DAPI), organelle specific trackers, etc. Furthermore, the presence of a transparent semi-permeable membrane enables optical detection from both top and bottom of the plate, i.e. both upright and inverted microscopy is possible.

The methods according to the invention enable seeding and cultivation of all cell types, comprising adherent and non-adherent cells. Non-adherent cells, for example, have in particular been difficult to order into single cell arrays due to the difficulty of tethering artifacts. Tethering artifacts for both adherent and non-adherent cells may however be avoided by using the inventive methods. The wells of the microplate used when performing the inventive methods provide suitable compartments for all cell types, comprising adherent and/or non-adherent cells. The wells can further be sealed with a semi-permeable top membrane. Accordingly, the problem of disruptive anchoring of cells will not arise in the inventive methods.

The methods of seeding and cultivation according to the invention can be used for any primary cells or cell lines. As non-limiting examples, the methods may be used for neoplastic cells, such as for example human cancer cells, and for stem cells. Successful seeding and cultivation of human leukemia cells, human myeloma cells, mouse embryonic stem cells and adult neural cells according to the inventive methods are presented in the appended Example. Seeding and cultivation of cancer cells, for example, enable rapid and controlled clinical testing.

When used for seeding and cultivation of stem cells, the inventive methods furthermore enable identification of stem cells within a cell population, monitoring of cell differentiation, screening of markers on single stem cells/clones, etc.

In an embodiment of the inventive method of cultivation, the analysis comprises analysis of cell response to the analytic medium. The cell response may for example be selected from cell viability, cell death, clone formation and cell resistance.

Individual and simultaneous analysis of several thousands of cells is enabled by the methods according to the invention. By controlled seeding of individual cells to predefined locations of the microplate, analysis of single cell heterogeneity and colony formation, for example in relation to drug sensitivity, can easily be accomplished.

Large cell samples comprising millions of cells are however not a requirement for performing the methods. On the other hand, both large and small cell samples can be used with the methods. Compared to previously known methods, the methods according to the invention can be performed using a lower number of cells. The methods are therefore well suited for, for example, screening of limited amounts of patient or primary cell samples. The methods according to the invention enable investigators and scientists to analyze characteristics of patient samples on a single cell level, potentially leading to a more optimized treatment. As shown in the appended example, cell consumption drastically decreases to 0.6% of the cells needed in conventional methods (Larsson, R., Kristensen, J., Sandberg, C. & Nygren, P. Laboratory determination of chemotherapeutic drug resistance in tumor cells from patients with leukemia, using a fluorometric microculture cytotoxicity assay (FMCA). Int J Cancer 50, 177-185 (1992)). Conventional methods often require cell samples comprising millions of cells, since analysis of for example cell sensitivity to drug addition is measured as an average of cell response for 50 000-100 000 cells.

In some embodiments of the inventive methods, at least two cells are seeded into at least one well of the microplate. By seeding two or more cells into one well of a microplate, or alternatively by seeding single cells, cell-to-cell interaction studies can be performed in a high-throughput manner. Another possibility is seeding of different types of cells into one well. Thus, controlled mixtures of different types of cells in different wells can be obtained.

The methods according to the invention can be used in diagnostics, research, drug discovery, agriculture, stem cell analysis, etc. In addition, the methods are suitable for use in clinical medicine, for identification of potential drug targets, optimization of a lead compound for a specific disease, test of cell viability, test of toxicity when cells are exposed to a specific compound, test of sensitivity to a specific compound, basic biological research, etc. The term compound refers to any chemical compound.

Moreover, the methods according to the invention can be used for studying cell response to drug gradients, cell morphology, temperature gradients, as well as electrical stimulation of the cells. The methods could also be one step towards individualized medicine and/or pharmacotherapy. Preclinical testing of drugs by the methods according to the invention could reduce the need of animal testing.

The inventive methods allow for similar detection and analysis techniques in a microplate as can be accomplished in any other microplate. However, the inventive methods enable detection and analysis for thousands of cells in parallel. In addition, the methods according to the invention enable real-time monitoring of changes over time of protein localization and metabolite contents, which can be done for thousands of single cells in parallel. The methods could be used for PCR in-plate, cell lysis, RNA/DNA analysis, protein detection, stem cell research and differentiation studies, etc.

The novel methods for high-throughput seeding and cultivation of single cells followed by single cell analysis have the potential to become important diagnostic tools when studying tumour cells from for example leukemia patients, with respect to e.g. heterogeneity, proliferation, apoptosis and sensitivity to chemotherapeutics at the single cell/clone level.

The methods according to the invention will now be described in a non-limiting manner by the following Figures and Example.

### BRIEF DESCRIPTION OF THE FIGURES

FIG. 1 is a drawing that illustrates an example of a method of cell cultivation according to the invention. Single cells from patient cell samples (1) are seeded by flow cytometry (2) into the microplate (3). The whole microplate is incubated (4). Analysis can be done instantly (3), during incubation (4) and/or after incubation (5).

FIG. 2 is a drawing that illustrates a microplate useful in the methods of seeding and cultivation according to the invention.

a) displays a microplate having four sections with different well sizes. A 128 mm×86 mm microplate having any of the displayed well sizes would contain 1536-6144 wells. Scale bar 1.5 cm.

b) is an enlarged section of a) displaying the microplate with different well sizes.

c) is an enlarged section of b) displaying the microplate with wells having tilted walls for facilitated cell seeding.

FIG. 3 is a photo displaying a fluorescently labeled single cell in one well directly after cell seeding.

FIG. 4 is a series of photos displaying clone formation in one well (day 1-14) starting with a single cell on day 1.

FIG. 5 is a series of photos displaying proliferation of leukemia cell samples from a patient in one well. The enlarged section shows two daughter cells after 4 days of cultivation.

FIG. 6 is a diagram showing percentage of dead cells after addition of lysis buffer (y-axis). The x-axis represents amount of lysis buffer in terms of the number of droplets added.

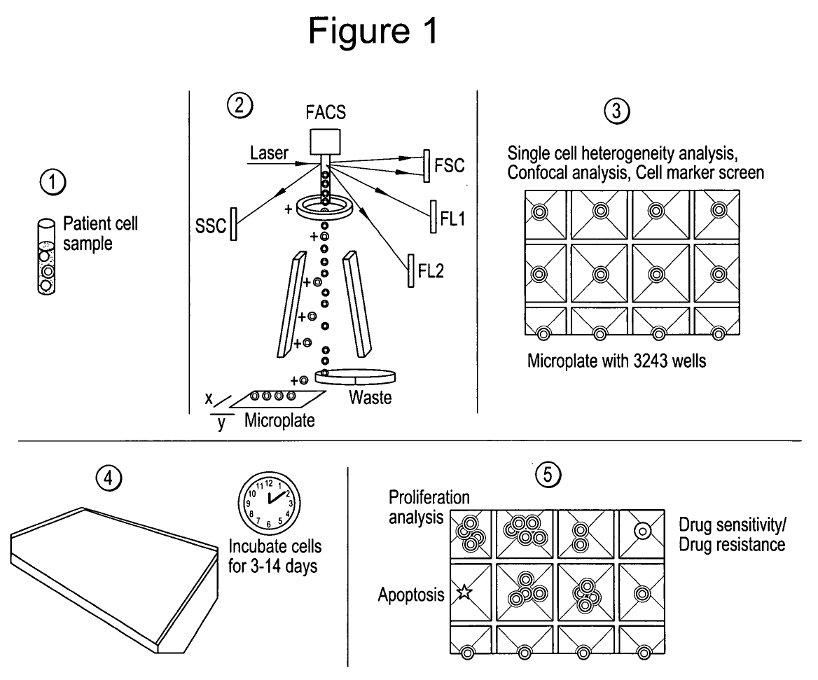

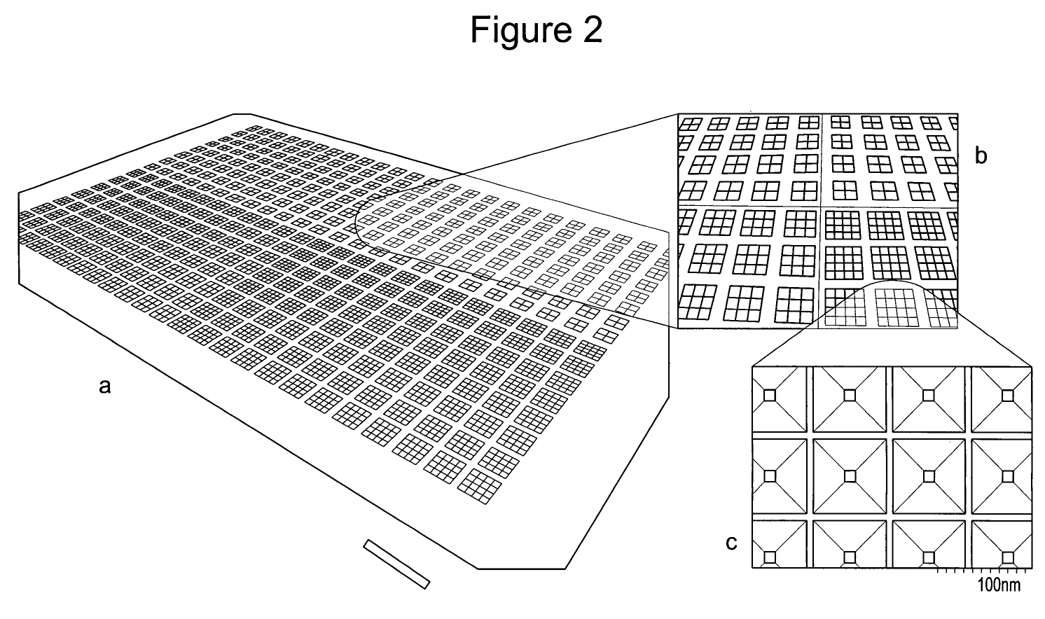

FIG. 7 is a series of photos displaying confocal images (a-e) of cell proliferation for a single embryonic stem cell (ES). Four antibodies were used for labeling of one single ES cell in one well after 1 (a-d) and 3 days of cultivation. Scale bar 10 μm.
a) displays labeling of DNA (corresponds to the grey portion of the image).
b) displays labeling of filamin (HPA ab, grey portion).
c) displays labeling of catreticulin (grey portion).
d) displays labeling of tubulin (grey portion).
e) displays an overlay image of an antibody-labeled cell colony according to a-d) after 3 days of single cell cultivation.

FIG. 8 is a photo displaying a differentiated adult neural stem cell in a well after 3 days of cultivation (upper right corner).

### EXAMPLE

In the following example, leukemic K-562 cells, leukemic patient cells and human myeloma cells were seeded and cultivated according to the inventive methods.

### Materials and Equipment

### The Microplate

The microplate used in all experiments below had a sandwich structure of three levels: a bottom glass plate, an etched silicon microgrid plate and a semi-permeable top membrane.

The microplate was produced with standard micro fabrication techniques involving nitride deposition, lithography, KOH silicon etching and nitride strip. The 500 μm thick silicon grid was anodically bonded to a 500 μm glass slide where after the plate was sawed into microtiter plate format (128 mm×86 mm). The wells of the microplate had tilted walls (54.7°) to facilitate cell seeding. The well bottom was 650×650 μm and the top opening was 1360×1360 μm. The total plate thickness was 1 mm and the centre-to-centre distance between wells was 1500 μm. The plate had 3243 wells of which 256 wells were used. The well volume was 500 nl.

### Semi-Permeable Top Membrane

Different semi-permeable membranes were evaluated for sealing of the wells, in order to optimize cell cultivation conditions and to prevent evaporation.

In total 9 membranes, sealing tapes or films were tested (table 1).

In general, evaporation tests and/or cell viability tests were performed for the different membranes. For the evaporation tests, cells were not necessarily seeded. For the cell viability tests, K562 cells were used. Growth medium and cells were simultaneously added to the wells of a microplate either manually by using a pipette or automatically by using a FACS. Thereafter, the wells were covered with the membrane and incubated in a cell incubator at 37° C., 5% CO₂ and elevated humidity. Cell clone formation was observed by light microscopy and cell viability was investigated by Trypan Blue staining.

Initially, 5 liquid-permeable hydrophilic membranes (1-5) were selected based on the idea that cultivation media could be exchanged during long cell cultivation periods. By having the cells in the bottom of the wells, and letting growth/cultivation media pass the filter membrane (permeable also to oxygen and carbon dioxide, necessary for cell cultivation), it was believed that cells could stay alive longer. After covering the wells with the membranes, the membranes were wetted by dropwise addition of a small amount of growth medium onto the membrane. Membranes 1-5 did not seal optimally onto the plate. In addition, the membranes dried up whereby cell cultivation was made impossible.

Therefore, 3 different gas-permeable (but not liquid permeable) membranes, or sealing films, were chosen for testing (6-8). These membranes were randomly chosen for their known suitability for cell cultivation in standard microtiter plates. However, for membrane no. 6, only 50% of the cells were alive after 45 h. Cell cultivation with membranes 7-8 resulted in 100% dead cells after 45 h.

Thus, the commercially available plastic membranes or films did not result in sufficient sealing on the silicon surface of the microgrid plate. Also, utilization of these membranes or films resulted in bubble formation in the wells and, above all, unsatisfactory cell viability.

Finally, a home-made membrane made out of PDMS was tested. The membrane was casted by mixing Sylgard® 184 Silicone Elastomer according to the manufacturer's instructions (Dow Corning). Mixing was performed manually with a pencil-like stirrer. The elastomer mix (total volume 10 ml) was then poured into a plastic lid (facing top-down, normally used for multi well plates, from Greiner bio-one). The thickness of the membrane was approximately 2-3 mm and the dimensions matched those of a standard microtiter plate. The plastic lid containing the elastomer mix was cured in 70° C. for 2 h. After a membrane had formed, it was peeled off the plastic lid, wrapped in aluminum foil and autoclaved (121° C. for 20 min). The membrane was kept sterile in the aluminum foil until being manually placed on top of the microplate. The resulting semi-permeable membrane was transparent. Before covering the microplate with the membrane, a few drops of growth culture media were placed on the microplate to create an excess of liquid and to thereby prevent air bubble formation within the wells.

A cell suspension was added manually to the microwells, whereby the plate was sealed with membrane no. 9. The plate was incubated in a cell incubator and after 72 h the viability was determined with Trypan Blue (dead cell stain). The membrane was removed, followed by addition of Trypan Blue to the microwells. After 5 minutes the dead cells stained blue, whereas the living cells stayed unstained. Detection was done by manually screening the plate, well by well, in a light microscope. The viability was very high, with only a few percent of dead cells. It was concluded that cell viability in the microplate was as high as in standard (petri dish) cell cultivation methods when compared to cell cultivation in a control petri dish. Cells had splitted into colonies (i.e. formed clones) which were observed with light microscopy in the same manner as described above.

In cross-comparative studies between the sandwich-structured microplate and other types of plates, i.e. conventional 24- and 384-well plates, and membranes 6 and 9, it was shown that both membrane types resulted in good cell viability for the conventional plates, whereas for the sandwich-structured microplate, membrane no. 6 gave low cell viability and membrane no. 9 gave very high cell viability. Cell viability was measured after 72 hours.

The same membranes were also compared in a viability study wherein single cell seeding was performed with a FACS. Again, in wells covered by membrane no. 6 no clone formation had taken place, whereas in wells covered by membrane no. 9 clone formation had taken place and differentiation was observed until 12 days after seeding.

Thus, a PDMS membrane was used in the following experiments.

TABLE 1

| | | Catalogue | Pore size |
|---|---|---|---|
| Membrane material | Supplier | no. | (μm) |
| 1 PVDF, hydrophilic | Millipore | HVLP14250 | 0.45 |
| 2 PVDF, hydrophilic | Millipore | GVWP14250 | 0.22 |
| 3 Polyethersulphone, | Millipore | GPWP14250 | 0.22 |
| hydrophilic | | | |
| 4 Mixed cellulose esthers, | Millipore | GSWP14250 | 0.22 |
| hydrophilic | | | |
| 5 Mixed cellulose esthers, | Millipore | HAWP14250 | 0.45 |
| hydrophilic | | | |
| 6 BreathEasy, hydrophobic | Diversified | BEM-1 | |
| | Biotech | | |
| 7 BreathEasier, hydrophobic | Diversified | BERM-2000 | |
| | Biotech | | |
| 8 Area Seal, hydrophobic | Web | WTS-7014 | |
| | Scientific | | |
| 9 Polydimethylsiloxane | In-house | | |

A way of further preventing evaporation during long cell seeding procedures would be to pause the seeding and cover the part of the microplate that has already been seeded with cells with a semi-permeable membrane, before continuing seeding the remaining parts of the plate.

### Apparatus for Cell Seeding

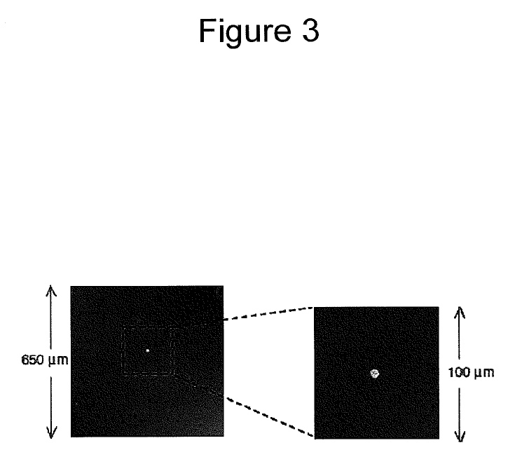

The first step towards long-term single cell analysis is to place a single cell into each well of the microplate, in a highly controlled and high-throughput manner. A FACS (FACSVantage SE Cell Sorter (BD Biosciences)) was used for performing the cell seeding. FIG. 3 demonstrates the precision of cell seeding using a FACS.

### Cell Seeding

The clean plates and semi-permeable top membranes were autoclaved for 20 min at 121° C. to obtain a sterile cell culture environment. An excess of recommended growth medium was added to the plate, and manually distributed evenly to all wells by an autoclaved hand scraper (VWR) or sterile cell scrapers (Falcon, for one-time use only). Then, automatic single cell seeding into predefined wells of the microplate was carried out using the FACS. A motorized x/y-stage combined with the software CloneCyt Plus (BD Biosciences) was used to control the exact position of every cell sorted in the plate. Selection of the cells to be seeded was performed based on their individual FSC/SSC (forward scatter/side scatter) properties (i.e. size and granularity). The cells were analyzed at a rate of approximately 100 cells/s. A representative set of cells was sought, meaning that all viable cells in the sample were selected for seeding into the plate. The cells were sorted directly from recommended growth medium in concentrations ranging from 10⁵-10⁶ cells/ml. FACS Clean solution (BD Biosciences) was let through the fluidics of the FACS prior to experiments to obtain sterility. The x/y-stage and the surrounding of the fluidics outlet was cleaned with 70% EtOH. During analysis, sterile 0.25×PBS was used as sheath buffer. After cell seeding, a semi-permeable membrane was applied to the top of the microplate to seal the wells. The plate, loaded with single cells, was incubated in a humidified atmosphere at 5% CO₂ and 37° C. for 3-14 days.

The time needed for seeding single cells was one second per well. The resulting well occupancy of single cells was approximately 97%, i.e. in approximately 97% of the wells, single cells had been seeded as intended. Fluorescence analysis was used for estimation of well occupancy.

### Leukemia Cells

Single leukemic K-562 cells (ECACC No. 89121407) were seeded as described above and cultured in the microplate in RPMI-1640+L-glut, supplemented with 10% Foetal Bovine Serum (GIBCO) and antibiotics/antimitotics in a humidified atmosphere, 5% CO₂, 37° C. up to 14 days. The humidity chambers consisted of home-made plate holders in plastic trays (Bio-Rad), where the bottom was covered with deionized water.

The K-562 cell line was cultured in parallel in standard petri dishes (BD Falcon), throughout the whole study.

Less than 5% of the wells per microplate were excluded from the study due to air bubble formation during sealing, which generally make cell detection impossible. The exclusion number was determined by visual inspection (if needed in a microscope) of fluorescently labeled K-562 cells for 256 neighbouring wells in 3 replicates followed by statistical calculation.

### Microscopy Analysis

The K-562 cells were fluorescently labeled with Calcein AM (Molecular Probes) according to the manufacturer's instructions. May-Grünewald/Giemsa (MGG) staining (VWR) of cells enabled observation of different types of cells. For viability tests, the Trypan exclusion method was used (Sigma). Cell proliferation observations were performed with an Olympus BX51 light microscope, outfitted with a manually adjustable x/y-stage and a digital camera (Olympus Camedia C-4000 zoom). Using a 10× objective lens, pictures were taken at various time points. Fluorescence imaging for studying cell seeding accuracy was performed with a Zeiss LSM 510 Meta confocal microscope equipped with motorized x/y-stage.

Leukemia Cell Samples from Patients

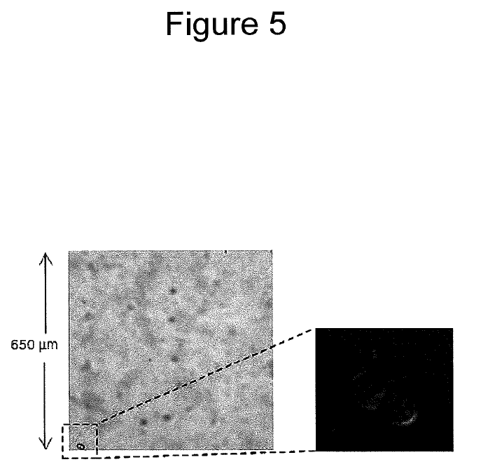

Patient cell samples were purified with Ficoll-Paque (GE Healthcare) at Uppsala Academic Hospital (Uppsala, Sweden) and delivered in frozen aliquots of 10⁵ cells/ml. The mononuclear cells were thawed, centrifuged and suspended in a recommended growth medium, followed by immediate seeding using FACS into the plate. The same FACS sorting protocol, including preparations and incubations, was used for all cell types. The leukemia cell samples from patients showed the ability to grow in-plate (FIG. 5).

### Proliferation Analysis

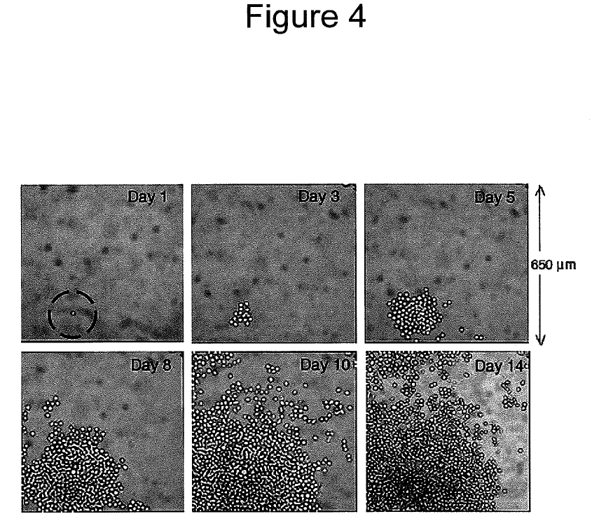

The next step, following single cell seeding, was clone formation analysis and study of proliferation in the plate. K-562 leukemia cells were successfully seeded and cultivated in the plate for up to 14 days (FIG. 4), which was followed by evaluation of viability and colony formation.

Cell viability in the microplate was comparable with viability in other cell cultivation systems. Single cell cultivation experiments in fifteen randomly chosen wells were terminated after 6 days, and cells were stained with the dead-cell dye Trypan Blue. This revealed an average of 95% viable cells in each well.

After two weeks of cultivation, up to 14 generations of cells were formed, which means that the initial single cells gave rise to more than 10 000 cells in each microwell. In addition, 256 neighbouring wells were followed during 6 days, which showed that 93% of all single K-562 cells gave rise to colony formation. The colony size differs, as expected, from well to well due to cell heterogeneity and non-synchronized cell line.

May-Grünewald/Giemsa (MGG) was used for staining in-plate. MGG is a stain for air-dry cytology preparations, commonly used for distinction between different blood cells. First, the membrane was peeled off the microplate, while still keeping the cells in their respective wells. The growth medium was evaporated to dry the cells in-plate. In some cases the whole plate was stored at −18° C. for later analysis, after drying of the cells. The plate was maintained in room temperature for a couple of minutes before staining. No differences in results between freezed and non-freezed samples in-plate were detected, which implies that cells can be analyzed directly (fresh) or thawed for this type of staining.

Furthermore, cell consumption was only 0.6% of the number of cells needed in conventional methods, see Larsson et al, Int J Cancer (1992) (supra).

### Human Myeloma Cells

Other cell lines that were cultivated in-plate were the human myeloma cell line RPMI8226 and the doxorubicin-resistant counterpart RPMI8226_{Dox40}. These cell lines have some adherent-like properties and tend to sit on the bottom of the well while proliferating.

The human myeloma cell lines RPMI8226 and RPMI8226_{Dox40} (ECACC No 87012702 and primary leukemic cells) were cultured in parallel in flasks (Nunc) in the same growth medium as the leukemia cells above (RPMI-1640+L-glut, supplemented with 10% Foetal Bovine Serum (GIBCO) and antibiotics/antimyotics in a humidified atmosphere, 5% CO₂,). 0.24 μg doxorubicin was added per ml RPMI8226_{Dox40} cell culture once a month to maintain the doxorubicin resistance of this cell line. The RPMI8226_{Dox40} cell line was trypsinized (GIBCO) before passages to remove cells tending to adhere from the surface of the culture flask.

### Addition of Drugs

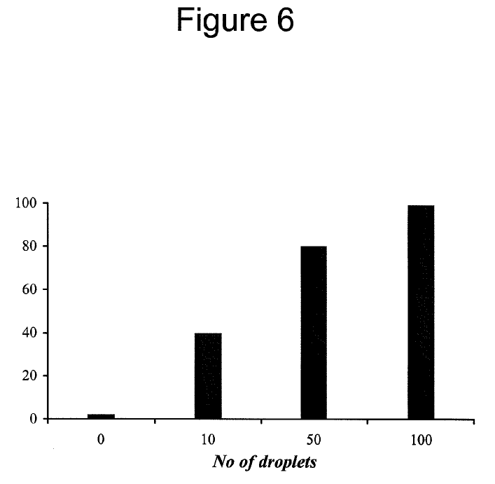

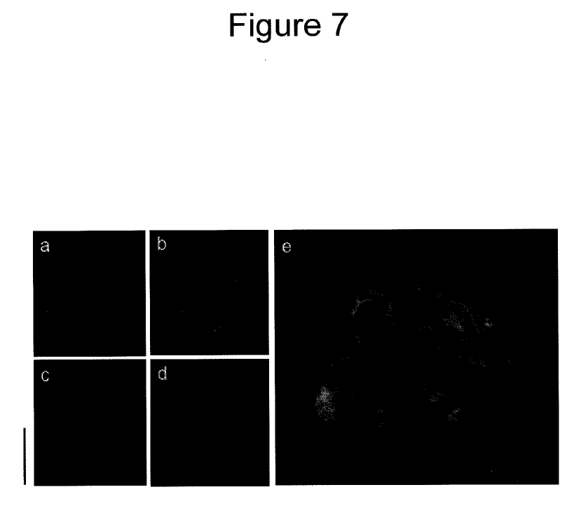

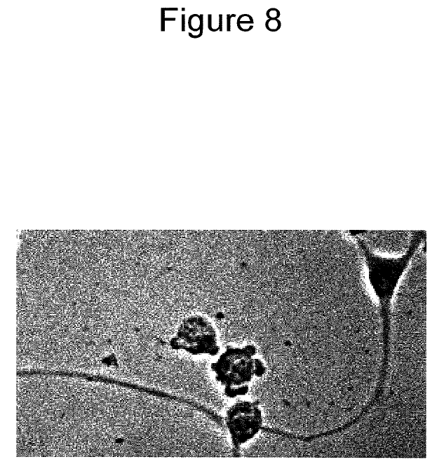

The FACS was used for addition of lysis buffer (0.1% Triton X-100) as a model drug substance in a proof-of-concept study. Drug addition, i.e. here, the addition of lysis buffer, was performed in the same manner as the cell seeding. Thus, a portion (droplet) of lysis buffer was added to chosen wells in a highly controlled manner, see FIG. 6. It was shown that the percentage of dead cells (y-axis) increased with an increased amount of lysis buffer (x-axis).

Another option for investigating cell response to drug addition is to seed several cell types and treat them similarly. This was done with the chemotherapeutic drug doxorubicin, and the results are shown in Table 2. Five microplates were seeded with two cell types: doxorubicin sensitive myeloma RPMI8226 cells and its doxorubicin resistant sub line RPMI8226_{Dox40}. Growth medium spiked with five different concentrations of doxorubicin was manually added to respective plate before cell seeding. Results after 6 days of incubation clearly showed that only resistant cells can proliferate at higher drug concentrations, demonstrating the capability of this single cell analysis methodology as a tool for drug resistance screening.

TABLE 2

| Doxorubicin [μM] | Sensitive (RPMI8226) | Resistant (RPMI8226_{Dox40}) |
|---|---|---|
| 10 | — | — |
| 0.2 | — | Cell growth |
| 0.02 | — | Cell growth |
| 0.002 | Cell growth | Cell growth |
| 0.000 | Cell growth | Cell growth |

### Screening for Stem Cell/Progenitor Markers on Single Cells

High throughput coating procedures for improved cultivation conditions were developed for embryonic stem (ES) cells and adult (primary) neural stem (NS) cells (Karolinska Institute, Stockholm, Sweden) as well as the three adherent cell lines U-2 OS (osteosarcoma), SH-SY5Y (neuroblastoma) and SK-BR-3 (adenocarcinoma), wherein the three latter were used as controls for adherent conditions. Single ES cells and NS cells were cultivated for 1-3 days. ES cells were also cultivated for one week.

For improved adherent cell culturing conditions, coating procedures for the whole plate were optimized (table 3) for mouse ES cells and mouse NS cells as well as the adherent human cell lines. The same coating protocol was used for the cell types and coatings according to table 3. Therefore, only coating of plates for cultivation of ES cells is described. Microplates of array format (76 mm×24 mm), each comprising 1081 wells, were used. The plate was pre-coated with 800 μl 0.2% gelatin solution. The gelatin solution was added to the plate and distributed evenly over the plate with a cell scraper (BD Falcon). The plate was left in room-temperature for 5-15 minutes. Thereafter the plate was rinsed with 3×5 ml sterile 1×PBS (phosphate buffer saline) by gently pipetting PBS over the entire plate. Lastly, the plate was quickly turned upside down on a tissue, to remove liquid from the wells. Cell viability in the coated plate was thereafter investigated by seeding and cultivating ES cells (in recommended growth medium). Here seeding was manually performed by pipetting cells into wells.

Poly-L-lysine (0.1 mg/ml) and fibronectin (10 ug/ml) were found to provide the best conditions for cell proliferation for NS cells and adherent cells respectively.

After establishing optimal coating conditions, single cell seeding and cultivation according to the inventive methods were performed in pre-coated plates. For ES cells, two plates of array format were coated according to the protocol above. Growth medium (recommended growth medium for ES cells) was then added to the wells of the plates. The same seeding protocol as for leukemia cells was used. One plate was incubated for 1 day (FIG. 7 a-d) and the other plate was incubated for 3 days (FIG. 7 e).

Protocols for fixation, antibody labeling and fluorescence imaging were developed for single stem cells/clones in-plate.

The plates were gently (not to remove adherent cells) washed with PBS, by either dipping the plates into a beaker containing PBS or by pipetting 2×5 ml PBS over each of the plates. Thereafter, ice-cold MeOH was added and the plates were placed in the freezer. After 5 minutes, the plates were removed from the freezer and washed by dipping the plates into a beaker containing 1×PBS. The plates were quickly turned upside down on a tissue to remove liquid from the wells. Primary antibody in blocking solution (4% fetal bovine serum (FBS) in PBS) was added. The plates were left overnight in 4° C.

The following day the plates were washed 3×5 minutes with 1×PBS in a beaker. Secondary (light sensitive) conjugated antibodies Alexa 488, Alexa 555 and Alexa 647 in blocking solution (4% FBS in PBS) were added and the plates were left for 1.5 hours in room temperature. 1 μl secondary antibody (antibody concentration 2 mg/ml) was added to 1 ml blocking solution. The plates were washed 2×3 minutes with PBS (in a plastic beaker). 0.300 μM nucleus-dye DAPI (1 μl DAPI in 1 ml PBS) was added and the plates were left for 4 minutes in room temperature. The plates were washed 3×5 minutes with PBS (in a plastic beaker), sealed with a membrane and put in the fridge.

Single ES cells were cultivated for from 1 to 3 days and showed good results on single cell proliferation. When clones had been formed, the fixation and antibody labeling protocol as described above was performed in-plate followed by fluorescence imaging (FIG. 7 a-e).

Adult NS cells were also cultivated (in recommended growth medium) in a microplate of array format with the aims of a) screening single stem cells in uncoated wells to find undifferentiated stem cells (neurosphere forming cells), and b) plating stem cells in coated wells and study cell differentiation (FIG. 8). Subsequently, NS cells were fixed and stained in-plate for a glial specific marker after 3 days of cultivation (not shown). In addition, weeklong ES cell cultivation was performed. For weeklong ES cell cultivation (not shown), a method for growth medium renewal was established. Thus, the membrane was removed and the plate was rinsed with new growth medium for facilitating long term cultivation of undifferentiated ES cells.

TABLE 3

| | Coating | | |
|---|---|---|---|
| | Poly-L-lysine | Fibronectin | Gelatin |
| Cell type | (0.1mg/ml) | (10 μg/ml) | (0.2% inPBS) |
| Embryonic Stem Cell | | | X |
| Adult Neural Stem Cell | X | | |
| U-2OS | | X | |
| SK-BR-3 | | X | |
| SH-SY5Y | | X | |

1. A method of cell seeding comprising the steps of:
providing a cell sample; and
seeding at least one cell of said cell sample into at least one well of a microplate comprising at least 5 wells/cm².

2. The method according to claim 1, wherein at least one single cell is seeded into at least one well.

3. The method according to claim 1, wherein said microplate comprises 5-700000 wells/cm².

4. The method according to claim 1, wherein said microplate has a well volume of from 1 pl to 200 μl.

5. The method according to claim 1, wherein the wells of said microplate have tilted walls.

6. The method according to claim 1, wherein the microplate comprises at least a bottom plate and a microgrid plate.

7. The method according to claim 1, further comprising sealing the microplate with a semi-permeable top membrane after cell seeding.

8. The method according to claim 1, wherein said seeding is performed using automatic robot equipment.

9. The method according to claim 8, wherein said seeding is performed using flow cytometry apparatus for fluorescence-activated cell sorting.

10. The method according to claim 1, wherein the cells are selected from the group consisting of neoplastic cells, human leukemia cells, human myeloma cells and stem cells.

11. A method of cell cultivation comprising the steps of:
providing a cell sample;
seeding at least one cell of said cell sample into at least one well of a microplate comprising at least 5 wells/cm²;
incubating the microplate; and
analyzing the contents of at least one well of the microplate.

12. The method according to claim 11, wherein at least one single cell is seeded into at least one well.

13. The method according to claim 11, wherein at least one of the steps of incubating and analyzing is repeated at least once.

14. The method according to claim 11, further comprising adding analytic medium to at least one well at any point after the seeding step.

15. The method according to claim 14, wherein at least one of the steps of incubating, analyzing and adding is repeated at least once.

16. The method according to claim 11, wherein cell seeding is performed using automatic robot equipment.

17. The method according to claim 16, wherein cell seeding is performed using flow cytometry apparatus for fluorescence-activated cell sorting.

18. The method according to claim 14, wherein adding analytic medium is performed using automatic robot equipment.

19. The method according to claim 18, wherein said equipment is flow cytometry apparatus for fluorescence-activated cell sorting.

20. The method according to claim 11, wherein seeding is performed first and the other steps are performed in any order.

21. The method according to claim 11, wherein seeding and analyzing are performed simultaneously.

22. The method according to claim 14, wherein the analytic medium comprises a compound selected from the group consisting of chemotherapeutic compounds, proteins, peptides, antibodies, affinity compounds, labeled compounds, nucleic acids, particles, and other compounds having or suspected of having a biological effect.

23. The method according to claim 14, wherein said analyzing comprises analyzing cell response to the analytic medium.

24. The method according to claim 11, wherein the cells are selected from the group consisting of neoplastic cells, human leukemia cells, human myeloma cells and stem cells.

25. The method according to claim 11, wherein at least two cells are seeded into at least one well of the plate in the seeding step.

26. The method according to claim 25, wherein said analyzing comprises studying cell-to-cell interactions.

27. The method according to claim 11, wherein said microplate comprises 5-700 000 wells/cm².

28. The method according to claim 11, wherein said microplate has a well volume of from 1 pl to 200 μl.

29. The method according to claim 11, wherein the wells of said microplate have tilted walls.

30. The method according to claim 11, wherein the microplate comprises at least a bottom plate and a microgrid plate.

31. The method according to claim 11, further comprising sealing the microplate with a semi-permeable top membrane after cell seeding.
